(19) United States
(12) Patent Application Publication
(10) Pub. No.: US 2025/0017967 A1
(43) Pub. Date: Jan. 16, 2025

(54) ANTI-THYMOCYTE GLOBULIN FOR IMMUNOMODULATION OF A SUBJECT WITH REGULATORY T CELLS

(71) Applicant: QUELL THERAPEUTICS LIMITED, London (GB)

(72) Inventors: Iain MCGILL, London (GB); Christopher James SHARPE, London (GB)

(21) Appl. No.: 18/711,861

(22) PCT Filed: Dec. 16, 2022
(86) PCT No.: PCT/GB2022/053277

§ 371 Date: May 20, 2024

(30) Foreign Application Priority Data
Dec. 17, 2021 (GB) 2118404.9

(51) Int. Cl. A61K 35/17; A61K 39/00; C07K 16/28

(52) U.S. Cl. CPC A61K 35/17; A61K 39/4611; A61K 39/4621; A61K 39/4631; A61K 39/46433; A61K 39/46434; C07K 16/2803; A61K 2239/38

(57) ABSTRACT

The present invention relates to the field of adoptive cell therapy (ACT), particularly to a method for conditioning a subject for treatment with a medicament comprising regulatory T cells (Tregs), the method comprising administering a low dose of anti-thymocyte globulin (ATG) to the subject about 8 weeks or less before administration of the medicament.

### FIELD OF THE INVENTION

The present invention relates to the field of adoptive cell therapy (ACT), particularly for the immunomodulation of a subject using regulatory T cells (Tregs). More particularly, the invention provides a method for conditioning a subject for treatment with Tregs, i.e. conditioning a subject who is about to receive a Treg therapy, such as a transplant recipient.

### BACKGROUND TO THE INVENTION

Adoptive transfer of cells (e.g. immune cells, such as T cells), i.e. adoptive cell therapy (ACT), is an attractive approach for generating desirable immune responses, such as an anti-tumour immune response or to suppress or prevent an unwanted immune response. ACT using T cells, both naturally occurring cells (e.g. tumour-reactive lymphocytes) and engineered cells (e.g. lymphocytes engineered to express conventional T cell receptors (TCRs) or chimeric antigen receptors (CARs)), has found utility in treating numerous diseases, particularly in the cancer setting.

CD4⁺FOXP3⁺ regulatory T cells (Tregs) are a lymphocyte subset that is essential for the maintenance of dominant immunological tolerance by inhibiting the function of various effector immune cell subsets, including myeloid cells such as macrophages and dendritic cells. In addition, Tregs are also known to promote tissue repair and regeneration. Tregs have been shown to be useful for inducing tolerance in transplant recipients and in the treatment of numerous autoimmune and inflammatory diseases, such as inflammatory bowel disease. The prospect of ameliorating immunopathology and re-establishing tolerance in inflammatory diseases has prompted a growing interest in the clinical development of Treg-based immunotherapies. However, for a Treg immunotherapy to be successful it is essential to provide a favourable microenvironment within the subject that supports the therapeutic activity of the cells.

It has been shown that conditioning a patient with one or more immunosuppressive chemotherapy drugs prior to T cell infusion (i.e. preconditioning the subject for the T cell treatment) can increase the effectiveness of the transplanted T cells. For example, in the cancer setting patients may receive cyclophosphamide and fludarabine as conditioning agents to reduce the number of endogenous lymphocytes and increase likelihood that the infused cells will engraft and/or reach their target in sufficient numbers to achieve a therapeutic effect.

Immunosuppressive chemotherapy drugs are also used in patients who receive transplants (e.g. solid organ transplants), as it is typical for such patients to require lifelong immunosuppression to prevent transplant rejection. A wide range of immunosuppressive chemotherapy drugs are known in the art and each of these drugs has its own adverse effect and toxicity profile that may result in serious morbidity or mortality. Moreover, transplant recipients often require other outpatient medications which are needed to maintain the integrity of the transplant and are very important in a patients' medication regimen. The interactions between these drugs can be complex and must be carefully managed.

Cyclophosphamide has been shown to be effective for conditioning cancer patients for treatment with anti-tumour T cells and its utility in this acute setting is particularly suitable due to its properties of selectively targeting rapidly diving cells and relatively short duration of T cell depletion. However, it is not typically used as an immunosuppressant in transplant recipients and its interactions with other drugs administered to transplant patients are not well characterised.

Thus, there is a need to develop clinical regimens that are able to maximise the therapeutic potential of ACT using Tregs, e.g. in the transplant setting.

### SUMMARY OF THE INVENTION

The present inventors have determined that adoptive cell therapy (ACT) using Tregs may be facilitated by conditioning patients with anti-thymocyte globulin (ATG) prior to administration of the Tregs. In particular, the inventors have determined that ATG can be used as a conditioning agent to achieve lymphocyte depletion in patients to create an environment particularly suitable for Treg survival and function in vivo, and may further ensure patient safety.

The use of ATG (particularly rabbit ATG (rATG)) in induction therapy in the setting of Liver Transplantation (LT) has increased over the past decade and has been shown to be advantageous for candidates with renal dysfunction in delaying the introduction of calcineurin inhibitors (CNIs), which are commonly associated with nephrotoxicity. Moreover, ATG has a tolerogenic effect by promoting the thymic export of de novo Tregs and expanding FOXP3⁺ T cells in vivo, while decreasing naïve T cells.

While not wishing to be bound by theory, it is expected that the profound and prolonged lymphodepletion caused by ATG allows for abrupt cessation of other immunosuppressive drugs, e.g. CNIs, that may otherwise have a negative effect on the infused Tregs. In particular, it is expected that the use of ATG allows for rapid withdrawal of tacrolimus, a CNI that would inhibit the expansion of the infused Tregs, and this in turn allows for the administration of only a single dose of infused cells. This is particularly advantageous because it reduces the number of Tregs required for administration, the production of which involves significant resources, and clinic time for the patient. As discussed in more detail below, the abrupt cessation of tacrolimus administration may be countered by a gentle tapering of other immunosuppressive drugs, such as everolimus, which does not affect Tregs significantly. Thus, it will be appreciated that the use of ATG as a conditioning agent may find particular utility in patients on an immunosuppressive therapy comprising a CNI, e.g. transplant recipients, particularly an immunosuppressive combination therapy consisting of tacrolimus and everolimus. Moreover, the use of ATG as a conditioning agent may find utility in a more general setting of treating inflammatory and autoimmune diseases.

Thus, in one aspect the invention provides a method for conditioning a subject for treatment with a medicament comprising regulatory T cells (Tregs), the method comprising administering a low dose of anti-thymocyte globulin (ATG) to the subject about 8 weeks or less before administration of the medicament.

In a further aspect the invention provides a method of immunomodulation of a subject in need thereof comprising the steps of:
(a) administering a low dose of anti-thymocyte globulin (ATG) to the subject; and
(b) administering a medicament comprising regulatory T cells (Tregs) to the subject,
wherein step (a) occurs about 8 weeks or less before step (b).

In another aspect, the invention may be viewed as providing a method for treating an inflammatory or autoimmune disease or condition in a subject comprising the steps of:
(a) administering a low dose of anti-thymocyte globulin (ATG) to the subject; and
(b) administering a medicament comprising regulatory T cells (Tregs) to the subject thereby treating the inflammatory or autoimmune disease or condition,
wherein step (a) occurs about 8 weeks or less before step (b).

In still another aspect, the invention provides a method for treating and/or preventing rejection of a transplant in a subject comprising the steps of:
(a) administering a low dose of anti-thymocyte globulin (ATG) to the subject; and
(b) administering a medicament comprising regulatory T cells (Tregs) to the subject thereby treating and/or preventing rejection of a transplant in the subject, wherein step (a) occurs about 8 weeks or less before step (b).

In yet another embodiment, the invention provides a method for inducing functional immune tolerance in a transplant recipient comprising the steps of:
(a) administering a low dose of anti-thymocyte globulin (ATG) to the transplant recipient; and
(b) administering a medicament comprising regulatory T cells (Tregs) to the transplant recipient thereby inducing functional immune tolerance in the transplant recipient,
wherein step (a) occurs about 8 weeks or less before step (b).

Alternatively viewed, the invention provides a low dose of anti-thymocyte globulin (ATG) for use in conditioning a subject for treatment with a medicament comprising regulatory T cells (Tregs), wherein said ATG is for administration about 8 weeks or less before administration of the medicament.

In a further aspect, the invention provides a low dose of anti-thymocyte globulin (ATG) for use in immunomodulation of a subject in need thereof, wherein said immunomodulation comprises administration of a medicament comprising regulatory T cells (Tregs) to the subject and wherein said low dose of ATG is for administration about 8 weeks or less before administration of said medicament.

In another aspect, the invention provides a low dose of anti-thymocyte globulin (ATG) for use in:
(a) treating an inflammatory or autoimmune disease or condition in a subject;
(b) treating and/or preventing rejection of a transplant in a subject; or
(c) inducing functional immune tolerance in a transplant recipient,
wherein (a), (b) and (c) comprises administration of a medicament comprising regulatory T cells (Tregs) to the subject/transplant recipient and wherein said low dose of ATG is for administration about 8 weeks or less before administration of said medicament.

In still a further aspect, the invention provides the use of a low dose of anti-thymocyte globulin (ATG) in the manufacture of a composition for conditioning a subject for treatment with a medicament comprising regulatory T cells (Tregs), wherein said composition is for administration about 8 weeks or less before administration of the medicament.

In yet another aspect, the invention provides the use of a low dose of anti-thymocyte globulin (ATG) in the manufacture of a composition for immunomodulation of a subject in need thereof, wherein said immunomodulation comprises administration of a medicament comprising regulatory T cells (Tregs) to the subject and wherein said low dose of ATG is for administration about 8 weeks or less before administration of said medicament.

In a further aspect, the invention provides the use of a low dose of anti-thymocyte globulin (ATG) in the manufacture of a composition for:
(a) treating an inflammatory or autoimmune disease or condition in a subject;
(b) treating and/or preventing rejection of a transplant in a subject; or
(c) inducing functional immune tolerance in a transplant recipient,
wherein (a), (b) and (c) comprises administration of a medicament comprising regulatory T cells (Tregs) to the subject/transplant recipient and wherein said composition is for administration about 8 weeks or less before administration of said medicament.

The invention also provides a combination therapy or product comprising a low dose of anti-thymocyte globulin (ATG) and a medicament comprising regulatory T cells (Tregs), wherein the components of the combination therapy or product are for separate and sequential use in immunomodulation of a subject, wherein said ATG is for administration about 8 weeks or less before administration of the medicament, optionally wherein said immunomodulation is for:
(a) treating an inflammatory or autoimmune disease or condition in a subject;
(b) treating and/or preventing rejection of a transplant in a subject; or
(c) inducing functional immune tolerance in a transplant recipient.

Alternatively viewed, the invention provides a kit comprising:
(i) anti-thymocyte globulin (ATG); and
(ii) a medicament comprising regulatory T cells (Tregs),
for separate and sequential use in immunomodulation of a subject, wherein said ATG is for administration at a low dose about 8 weeks or less before administration of the medicament,
optionally wherein said immunomodulation is for:
(a) treating an inflammatory or autoimmune disease or condition in a subject;
(b) treating and/or preventing rejection of a transplant in a subject; or
(c) inducing functional immune tolerance in a transplant recipient.

### DESCRIPTION OF FIGURES

FIG. 1 shows a graphical representation of the methods of the invention (e.g. the conditioning method (e.g. W-14 to W-4) and the immunomodulation method (e.g. W-14 to W30)) based on the representative protocol set out in Example 1.

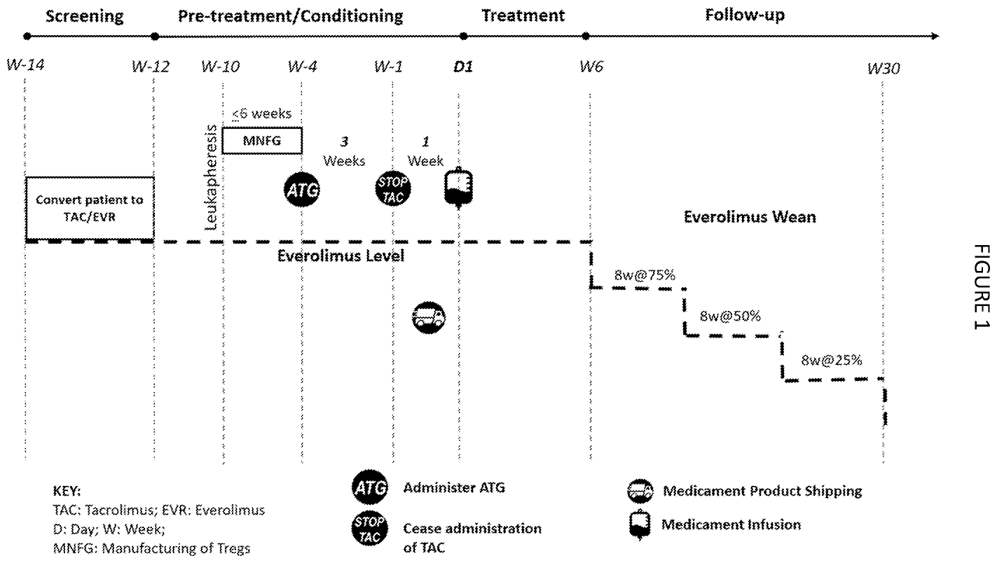

FIG. 2 shows a further graphical representation of the methods of the invention based on the representative protocol set out in Example 1. A product candidate comprising stable and suppressive natural Treg (nTreg) cells engineered with 3 genes is infused in the method. mTOR WEAN refers to weaning off an mTOR inhibitor immunosuppressive (IS). “B” indicates timepoints at which a biopsy is obtained.

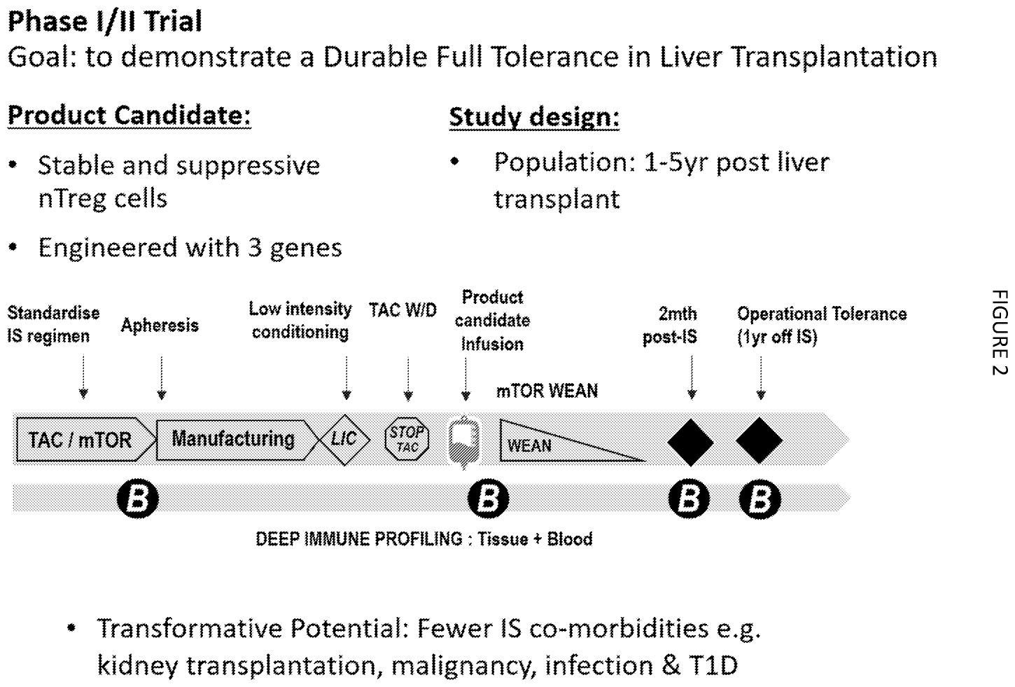

### DETAILED DESCRIPTION

The term “conditioning” as used herein refers to the process of preparing a subject (i.e. patient) who is about to receive a therapy for that therapy. Thus, it refers to altering the state of the subject to improve the efficacy of the therapy they are about to receive, e.g. to make the subject more receptive to the therapy. Thus, conditioning may be viewed as “preconditioning” the patient for treatment and these terms may be used interchangeably herein. Alternatively viewed, the process of conditioning a subject may be viewed as the first part of a multipart therapeutic regimen to treat and/or prevent a pathological state (e.g. to prevent the deterioration of an existing pathological state). In the method of the present invention, a low dose of anti-thymocyte globulin (ATG) is administered as, or as part of, the conditioning regimen to prepare the subject for treatment with a medicament comprising regulatory T cells (Tregs).

Whilst not wishing to be bound by theory, it is postulated that the use of ATG functions to deplete or reduce cells (e.g. lymphocytes, such as T cells) and/or other molecules (e.g. cytokines) that may function to interfere or inhibit the activity of the therapeutic Tregs (i.e. the medicament) to be infused into the subject. Alternatively viewed, ATG functions to achieve an environment within the subject that is particularly suitable for Treg survival and function. Thus, in some aspects, ATG may be seen as enhancing the in vivo environment for Treg survival and function. Alternatively viewed, ATG may function to enhance, improve or potentiate the effect of the Treg medicament. Thus, in some embodiments, conditioning may be viewed as enhancing, improving or potentiating the effect of the Treg medicament.

Anti-thymocyte globulin (ATG) refers to anti-human thymocyte immunoglobulin (lgG), typically obtained from rabbit or horse. Commercial pharmaceutical preparations of ATG are available from Genzyme™ (rabbit ATG (rATG)) and Pfizer™ (equine ATG (eATG)). In some embodiments, the ATG used in the methods and uses of the invention is rATG.

ATG is a selective immunosuppressive agent that acts primarily on T lymphocytes and their precursors. It is thought that the immunosuppressive effect of ATG arises from lymphocyte depletion, which is both peripheral and central; peripheral lymphocyte depletion can be detected as early as 24 hours after the first infusion. Lymphocyte depletion has been shown to occur in vitro by a number of different mechanisms including apoptosis, complement dependent lysis and antibody dependent cytotoxicity. Notably, ATG is used as an immunosuppressive agent in the context of solid organ transplants. It is thought that the combination of T cell depletion activity and down modulation of adhesion molecules results in interference with multiple pathways by which transplant rejection occurs. ATG typically is used in the treatment of renal allograft rejection and as an immunosuppressive agent before and/or during kidney transplantation.

As noted above, the inventors have determined that low dose ATG (e.g. low dose rATG) may find utility in conditioning subjects that are expected to benefit from treatment with Tregs, i.e. low dose ATG is particularly useful for preparing subjects for treatment with Tregs. Thus, in the context of the present invention, ATG (i.e. low dose ATG) may be viewed as a conditioning or preconditioning agent. Alternatively, in view of its principal activity, ATG may be viewed as a lymphodepleting agent.

A “low dose” of ATG refers to a dose that is substantially lower than the total cumulative dose typically used in the treatment of renal allograft rejection, which is about 10.5-21 mg/kg (e.g. about 1.5 mg/kg/day for 7 to 14 days). By substantially lower is meant at least about 40% lower than the cumulative dose typically used, e.g. about 45%, 50%, 55%, 60% or more lower than the cumulative dose typically used. It will be evident that the precise dose may depend on numerous parameters such as age and general condition of the subject, mode of administration, weight, body surface area, sex, other drugs being administered concurrently and the like. Thus, it may not be possible to specify an absolute “low dose” that is applicable to all subjects. The low dose for a particular subject may be determined relative to the dose that would have been administered to the subject if used to treat renal allograft rejection. In particular, a low dose refers to the amount of ATG (e.g. rATG) suitable to condition the subject for treatment with a medicament comprising Tregs.

Thus, in absolute terms, a low dose may refer to a total cumulative dose of about 6 mg/kg (mg of ATG/kg of subject) or less, such as about 5.5 mg/kg or less, about 5.0 mg/kg or less, about 4.5 mg/kg or less, about 4.0 mg/kg or less, about 3.5 mg/kg or less or about 3.0 mg/kg or less. Thus, a low dose of ATG (e.g. rATG) may be about 0.5-6.0 mg/kg, about 1.0-5.0 mg/kg, about 1.5-4.5 mg/kg, or about 2.0-3.5 mg/kg, such as about 1.5, 2.0, 2.5 or 3.0 mg/kg. In particular, a low dose of ATG (e.g. rATG) may be about 2.5 mg/kg.

It will be evident that the low dose of ATG may be administered over any period of time suitable to achieve the conditioning effect on the subject. The skilled person readily could determine a suitable dosage regimen, e.g. a dose of 1 mg/kg/day for 1-6 days, e.g. 2 or 3 days. However, in preferred embodiments, the low dose of ATG is administered in a single dose, such as a single dose of about 1.5-4.5 mg/kg, or about 2.0-3.5 mg/kg, such as about 1.5, 2.0, 2.5 or 3.0 mg/kg.

Advantageously, the low dose of ATG is calibrated to achieve the conditioning effect such that further doses of ATG are not required. In this respect, it will be understood that administering ATG after the medicament comprising Tregs is administered will affect the therapeutic efficacy of the medicament, e.g. the ATG will cause depletion of the administered Tregs. Thus, it is preferred that the subject does not receive any further doses of ATG after administration of the medicament comprising Tregs, i.e. during the period of treatment. In this respect, it will be evident that this does not preclude the possibility that the subject may be administered ATG after the end-point of the Treg therapeutic regimen described herein, e.g. in the course of treating a different disease or wherein the Treg therapeutic regimen described herein is repeated.

The low dose of ATG may also be calibrated to minimize the lymphodepleting effect on the Tregs administered to the subject, i.e. such that when the medicament comprising Tregs is administered the level of ATG in the subject does not substantially affect the efficacy of the administered Tregs. In this respect, the low dose of ATG is administered before administration of the medicament comprising Tregs, i.e. up to about 8 weeks before administration of the medicament comprising Tregs. Alternatively viewed, administration of the medicament comprising Tregs is deferred or delayed by up to about 8 weeks after administration of the low dose of ATG.

Thus, the timing of the administration of the Tregs may be modified based on the dose of ATG administered to the subject. For example, where a subject is administered a low dose of ATG at the higher end of the ranges described above, it may be advantageous to defer administration of the Tregs for the maximum period specified herein, e.g. about 8 weeks.

Similarly, the timing of the administration of the low dose of ATG may be modified based on the availability of Tregs. As discussed below, the Tregs may be derived from the subject to be treated and may be expanded and/or modified (e.g. engineered to express a chimeric receptor) prior to administration. As this process may take several weeks, administration of the low dose of ATG may be delayed to ensure that the Tregs are available for administration within a suitable window, e.g. within 8 weeks or less of administration of the low dose of ATG. Alternatively, if Tregs are available for administration immediately (e.g. allogenic Tregs), administration of the low dose of ATG may be performed as soon as possible to minimize the delay in treatment, i.e. so that the Tregs may be administered as soon as possible, e.g. within about 8 weeks or less of administration of the low dose of ATG. As discussed further below, it may be necessary to prepare the subject for administration of the low dose of ATG, e.g. by switching the calcineurin inhibitor therapy of the subject.

Thus, the low dose of ATG is administered to the subject about 8 weeks or less (e.g. up to about 8 weeks) before administration of the medicament comprising Tregs. As noted above, the timing of administration may depend on numerous factors and thus “about 8 weeks” may be viewed as including the range 6-10 weeks. In preferred embodiments, the low dose of ATG is administered to the subject about 2-8 weeks, 3-7 weeks, 3-6 weeks or 3-5 weeks, e.g. about 4 weeks, before administration of the medicament comprising Tregs.

“Regulatory T cells (Tregs)” or “T regulatory cells” are immune cells with immunosuppressive function that control cytopathic immune responses and are essential for the maintenance of immunological tolerance. As used herein, the term Treg refers to a T cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs. Antigen-specific Tconv cells co-cultured with Tregs as described herein may proliferate 5%, 10%, 20%, 30%, 40%, 50%, 60%, 70%, 90%, 95% or 99% less than the same Tconv cells cultured in the absence of Tregs as described herein.

Antigen-specific Tconv cells co-cultured with Tregs may express at least 10%, at least 20%, at least 30%, at least 40%, at least 50%, or at least 60% less effector cytokine than corresponding Tconv cells cultured in the absence of Tregs. The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor).

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7 (10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

A Treg may have a demethylated Treg-specific demethylated region (TSDR). The TSDR is an important methylation-sensitive element regulating FOXP3 expression (Polansky, J. K., et al., 2008. European journal of immunology, 38 (6), pp. 1654-1663).

Different subpopulations of Tregs are known to exist, including naïve Tregs (CD45RA⁺FOXP3^{low}), effector/memory Tregs (CD45RA⁻FOXP3^{high}) and cytokine-producing Tregs (CD45RA⁻FOXP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc., expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA+. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100-fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127^{low} can be used to determine whether a cell is a naïve Treg. Methods of determining whether isolated cells are naïve Tregs or have a desired phenotype can be carried out as discussed below in relation to additional steps which may be carried out as part of the invention, and methods for determining the presence and/or levels of expression of cell markers are well-known in the art and include, for example, flow cytometry, using commercially available antibodies.

Thus, the methods and uses of the invention relate to the use of a medicament comprising a plurality of Tregs as defined above, e.g. a population of Tregs. Thus, in some embodiments, the medicament comprises a population of Tregs. It will be appreciated that not all cells within a cell population may express the Treg markers described above to the same extent. Thus, a population of Tregs may comprise distinct and identifiable sub-populations of Tregs as defined above. For utility in the methods and uses of the invention, it may be sufficient that at least about 50% of the cells in the population are identifiable as Tregs, preferably at least about 60, 70, 80, 90 or 95% of the population are identifiable as Tregs, preferably naïve Tregs.

The Tregs for use in the invention may be modified or engineered Tregs. An “engineered cell” or “engineered Treg” as used herein means a cell which has been modified to comprise or express a polynucleotide that is not naturally encoded by the cell. It will be appreciated that Tregs may be engineered to express numerous polynucleotides to facilitate their use in the invention. For instance, the Treg may be engineered to express a chimeric receptor, e.g. a chimeric antigen receptor (CAR), that enhances the utility of the Treg in one or more uses described herein, e.g. that makes the engineered Treg more effective at treating and/or preventing a disorder as described herein than a corresponding Treg not expressing the chimeric receptor. Additionally or alternatively, the Treg may be engineered to express a polypeptide that improves other properties of the Treg, e.g. a polypeptide that improves the cryopreservation of the cell, functions to maintain the identity of the cell, functions to improve the proliferation of the cell and/or in vivo persistence, functions to induce cell death in response to stimulus, e.g. a safety switch polypeptide etc. For instance, the engineered Treg may comprise an exogenous polynucleotide encoding FOXP3 or an IL2 receptor polypeptide or portion thereof. In a representative example, the engineered Treg may be a Treg as described in any one of WO 2020/044055, WO 2021/170666, WO 2021/239812, WO 2019/202323, WO 2021/079149 (all of which are herein incorporated by reference) or may be engineered as described in any of the aforementioned applications or a combination thereof. Thus, in some embodiments, the Treg is a CAR-Treg, i.e. a Treg engineered to express a CAR.

The medicament of the present invention therefore may comprise a cell population comprising an engineered Treg cell (a plurality of engineered Treg cells). The cell population may have been transduced with a vector encoding a polynucleotide that is not naturally encoded by the cell. A proportion of the cells of the cell population may express the polynucleotide. Thus, where the engineered Treg comprises a polynucleotide encoding a chimeric receptor, e.g. a CAR, a proportion of the cells of the cell population may express the chimeric receptor, e.g. CAR, at the cell surface. Furthermore, a proportion of the cells of the cell population may co-express a chimeric receptor, e.g. CAR, and a further polypeptide (e.g. an accessory protein) as described above, e.g. exogenous FOXP3, safety switch etc. Thus, it will be appreciated that not all cells within a cell population may express the polynucleotide(s) that is (are) not naturally encoded by the cell, e.g. chimeric receptor. Particularly, at least 50, 60, 70, 80, 90, 95 or 99% of cells express the polynucleotide(s) that is (are) not naturally encoded by the cell, e.g. chimeric receptor.

The term “chimeric receptor” refers to a receptor protein comprising linked domains from two or more proteins, e.g. an exodomain from a first protein and an endodomain from a second protein. Typically, at least one of the domains is derived from a receptor protein. Thus, a chimeric receptor may be viewed as an “engineered receptor” and these terms are used interchangeably herein.

A chimeric receptor may comprise linked domains on a single polypeptide chain (a single contiguous chain) or may comprise two or more polypeptide chains (a multichain chimeric receptor), wherein at least one of the polypeptide chains comprises linked domains from two or more proteins. Thus, a chimeric receptor may comprise at least two polypeptide chains which may associate with each other when co-expressed, particularly through their transmembrane domains, and/or through an alternative dimerization site. Typically, each polypeptide chain within a multichain chimeric receptor will comprise of two or more linked domains, for example, a first polypeptide chain may comprise an extracellular domain and a transmembrane domain, and a second polypeptide may comprise a transmembrane domain and an endodomain, or a first polypeptide may comprise an extracellular domain, a transmembrane domain and an endodomain and a second polypeptide may comprise a transmembrane domain and an endodomain. However, it is also possible for one of the polypeptide chains to only comprise a single domain, typically an endodomain. It will therefore be appreciated that a chimeric receptor for use in the invention may comprise more than one of a particular domain within the same or within different polypeptide chains. For example, where the chimeric receptor is a multichain chimeric receptor, the chimeric receptor may comprise two transmembrane domains and/or two endodomains which may be the same or different.

A “Chimeric antigen receptor”, “CAR” or “CAR construct” refers to engineered receptors which can confer an antigen specificity onto cells (e.g. immune cells, such as Tregs). As discussed above, a CAR may comprise a single polypeptide chain or may comprise two or more polypeptide chains (e.g. a first polypeptide chain and a second polypeptide chain). In particular, a CAR enables a cell to bind specifically to a particular antigen, e.g. a target molecule such as a target protein, whereupon a signal is generated by the endodomain (comprising an intracellular signalling domain) of the CAR, e.g. a signal resulting in activation of the cell. CARs are also known as artificial T-cell receptors, chimeric T-cell receptors or chimeric immunoreceptors. Thus, the chimeric receptor for use in the invention may function to confer Tregs expressing the receptor with the ability to bind specifically to ligands associated with the conditions and disorders to be treated according to the invention.

The structure of CARs is well-known in the art and several generations of CARs have been produced. For instance, as a minimum a CAR may contain an extracellular antigen-specific targeting region, antigen binding domain or ligand binding domain, which is or forms part of the exodomain (also known as the extracellular domain or ectodomain) of the CAR, a transmembrane domain, and an intracellular signalling domain (which is or is comprised within an endodomain). However, the CAR may contain further domains to improve its functionality, e.g. one or more co-stimulatory domains to improve T cell proliferation, cytokine secretion, resistance to apoptosis, and in vivo persistence. As discussed above, the CAR may comprise more than one polypeptide chain and thus the domains may occur within the same or within different polypeptides, typically which associate with one another. Thus, a CAR may comprise two polypeptides wherein the first polypeptide comprises the extracellular domain, a transmembrane domain and optionally an endodomain, and the second polypeptide comprises an endodomain and optionally a transmembrane domain. Particularly, at least one endodomain in a multichain CAR will comprise an intracellular signalling domain.

Thus, a CAR construct generally comprises an antigen or ligand binding domain, optionally a hinge domain, which functions as a spacer to extend the antigen or ligand binding domain away from the plasma membrane of the cell (e.g. immune cell, e.g. Treg) on which it is expressed, a transmembrane domain, an intracellular signalling domain (e.g. the signalling domain from the zeta chain of the CD3 molecule (CD3ζ) of the TcR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g. within two polypeptides which associate with one another.

As discussed herein, the methods of the invention find particular utility in the treatment of transplant recipients, particularly liver transplant recipients. In this respect, antigens associated with organ transplants and/or cells associated with transplanted organs include a HLA antigen present in the transplanted organ but not in the patient, an organ-specific, tissue-specific or cell-specific antigen (e.g. a liver-specific antigen) or an antigen whose expression is up-regulated during transplant rejection such as CCL19, MMP9. SLC1A3, MMP7, HMMR, TOP2A, GPNMB, PLA2G7, CXCL9, FABP5, GBP2, CD74, CXCL10, UBD, CD27, CD48, CXCL11. Thus, in some embodiments, the engineered Treg for use in the invention comprises a chimeric receptor (e.g. a CAR) that selectively binds to a HLA antigen, e.g. HLA-A2, present in the transplanted organ but not in the patient, an organ-specific antigen (e.g. a liver-specific antigen such as Na+/taurocholate cotransporting polypeptide (NTCP)), a tissue-specific antigen, a cell-specific antigen, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10. UBD, CD27, CD48, CXCL11. In a representative example, the engineered Treg for use in the invention comprises a HLA CAR as described in WO 2018/001874 or WO 2020/201230 (which are both herein incorporated by reference).

Methods for engineering cells are known in the art and include, but are not limited to, genetic modification of cells, e.g. by transduction such as retroviral or lentiviral transduction, transfection (such as transient transfection-DNA or RNA based) including lipofection, polyethylene glycol, calcium phosphate and electroporation. Any suitable method may be used to introduce a nucleic acid molecule into a Treg cell. Non-viral technologies such as amphipathic cell penetrating peptides may be used to introduce a nucleic acid molecule into a Treg cell for use in the present invention.

Accordingly, an engineered cell (i.e. Treg) is a cell which has been modified e.g. by transduction or by transfection. Suitably, an engineered cell is a cell which has been modified or whose genome has been modified, e.g. by transduction or by transfection. Suitably, an engineered cell is a cell that has been modified or whose genome has been modified by retroviral transduction. Suitably, an engineered cell is a cell which has been modified or whose genome has been modified by lentiviral transduction.

As used herein, the term “introduced” refers to methods for inserting foreign DNA or RNA into a cell and includes both transduction and transfection methods. Transfection is the process of introducing nucleic acids into a cell by non-viral methods. Transduction is the process of introducing foreign DNA or RNA into a cell via a viral vector. Engineered Treg cells according to the present invention may be generated by introducing DNA or RNA, e.g. encoding a polypeptide (e.g. chimeric receptor), by one of many means including transduction with a viral vector, transfection with DNA or RNA. Cells may be activated and/or expanded prior to, or after, the introduction of a polynucleotide, for example by treatment with an anti-CD3 monoclonal antibody or both anti-CD3 and anti-CD28 monoclonal antibodies. Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The Treg of the medicament may be derived from a patient, e.g. a subject to be treated. Thus, the Treg cell population may be ex vivo patient-derived cell population. As described in more detail below, the cell may have been removed from a subject, optionally transduced or transfected ex vivo with a vector to provide an engineered cell, and expanded and formulated into a medicament prior to administration to the subject. Alternatively, the Treg may be a donor cell, for transfer to a recipient subject, or from a cell line, e.g. a Treg cell line. The cell may further be a pluripotent cell (e.g. an iPSC) which may be differentiated to a Treg prior to formulation in to the medicament.

Thus, the Treg cells (e.g. Treg cell population) of the medicament may be allogenic or autologous to the subject to be treated.

The term “medicament” refers to a pharmaceutical composition and these terms may be used interchangeably herein. Thus, a medicament is a composition that comprises or consists of a therapeutically effective amount of a pharmaceutically active agent. In the context of the present application the term “medicament” typically refers to a composition that comprises or consists of a therapeutically effective amount of a Treg cell or Treg cell population described herein. The term “pharmaceutical composition” typically refers to a composition that comprises or consists of a therapeutically effective amount of a non-cellular pharmaceutically active agent, e.g. ATG, immunosuppressive drug (e.g. mTOR inhibitor or calcineurin inhibitor (CNI)) such as everolimus or tacrolimus. However, it will be appreciated that a “medicament comprising Tregs” may alternatively be viewed as a “Treg composition”.

A medicament or pharmaceutical composition preferably includes a pharmaceutically acceptable carrier, diluent or excipient (including combinations thereof). Acceptable carriers or diluents for therapeutic use are well-known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The medicaments and pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the pharmaceutically active agent (e.g. Treg) and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The medicament and/or pharmaceutical composition may be administered in a manner appropriate for the therapeutic purpose described herein, e.g. for conditioning the subject for treatment and/or treating and/or preventing the disease or condition described herein.

While the quantity and frequency of administration of the medicament and/or pharmaceutical composition will be determined by such factors as the condition of the subject, and the type and severity of the subject's disease, as noted above, the inventors have determined that conditioning the subject using ATG advantageously enables the administration of a single dose of medicament comprising Tregs. Thus, in some embodiments, the subject is administered a single dose of the medicament. The medicament and pharmaceutical composition may be formulated accordingly.

The medicament and/or pharmaceutical composition can be administered via any suitable means. For instance, the medicament comprising Tregs and/or the pharmaceutical composition comprising ATG may be administered parenterally, for example, intravenously, or they may be administered by infusion techniques. The medicament and/or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The medicament and/or pharmaceutical composition may be formulated accordingly. The preparation of suitable parenteral formulations under sterile conditions is readily accomplished by standard pharmaceutical techniques well-known to those skilled in the art.

The medicament comprising Tregs and/or the pharmaceutical composition comprising ATG may be formulated in infusion media, for example sterile isotonic solution. The pharmaceutical composition may be enclosed in ampoules, disposable syringes or multiple dose vials made of glass or plastic.

It will be appreciated that other therapeutically active agents described herein, e.g. immunosuppressive drugs (e.g. mTOR inhibitors and CNIs, such as everolimus or tacrolimus), may be administered in their typical formulation and/or dosage range unless specified otherwise. Thus, for example, immunosuppressive drugs (e.g. mTOR inhibitors and CNIs, such as everolimus or tacrolimus), may be administered enterally, e.g. orally.

The medicament and/or pharmaceutical composition may further comprise one or more active agents.

Depending upon the disease and subject to be treated, as well as the route of administration, the medicament comprising Tregs may be administered at varying doses (e.g. measured in cells/kg or cells/subject). The physician in any event will determine the actual dosage which will be most suitable for any individual subject and it will vary with the age, weight and response of the particular subject. Typically, however, for Tregs, doses of 5×10⁷ to 3×10⁹ cells, or 10⁸ to 2×10⁹ cells per subject may be administered.

The cells may be appropriately modified for use in a medicament. For example, Tregs may be cryopreserved and thawed at an appropriate time, before being infused into a subject.

As noted above, the Tregs may be autologous to the subject to be treated, i.e. the Tregs for use in the preparation of the medicament may be obtained from the subject to be treated. Thus, the method of the invention may comprise a step of obtaining Tregs from the subject to be treated for use in the preparation of the medicament comprising Tregs. Any suitable method for isolating Tregs from the blood or a blood sample of the subject may be used, e.g. leukapheresis. It will be appreciated that the blood sample comprising the Tregs to be isolated should be obtained prior to lymphodepletion of the subject, i.e. prior to administration of low dose ATG. Thus, leukapheresis should be performed prior to lymphodepletion of the subject, i.e. prior to administration of low dose ATG.

Thus, the method of the invention may comprise a step of obtaining a blood sample comprising T cells (e.g. Tregs) from the subject or obtaining T cells (e.g. Tregs) from the subject prior to the step of administering low dose ATG to the subject, wherein the blood sample and/or T cells are for use in the preparation of the medicament comprising Tregs.

In a particular embodiment, the invention may comprise a step of performing leukapheresis on the subject to isolate T cells for use in the preparation of the medicament before the step of administering the low dose of ATG.

As blood samples and components thereof, e.g. T cells (e.g. Tregs) may be preserved and stored, e.g. cryopreserved, it will be appreciated that the blood sample may obtained from the subject and/or leukapheresis may be performed on the subject at any suitable time before the step of administering the low dose of ATG. In particular, the blood sample may be obtained from the subject and/or leukapheresis may be performed on the subject to provide sufficient time to prepare the medicament comprising Tregs. Conveniently, leukapheresis may be performed at least about 4 weeks before the step of administering the low dose of ATG, e.g. about 3-10 weeks, 4-8 weeks or a 5-7 weeks (e.g. 6 weeks) before step of administering the low dose of ATG.

As discussed above, Adoptive Cell Therapy (ACT), i.e. adoptive cell transfer of immune cells (e.g. Tregs, such as engineered Tregs) is an attractive approach for generating desirable immune responses, i.e. to modulate the immune system, particularly to suppress or prevent an unwanted immune response. Thus, the method of conditioning of the invention finds particular utility in methods for immunomodulation of a subject, i.e. in immunotherapy particularly ameliorating an immunopathology and re-establishing tolerance in inflammatory and autoimmune conditions. It will be appreciated that numerous conditions and disorders that involve an immunopathology or require control or management of the immune system may benefit from administration of a medicament comprising Tregs as described herein. In particular, it has been determined that administration of Tregs to transplant recipients may help to treat and/or prevent rejection of the transplant and/or induce functional immune tolerance in a transplant recipient. Thus, the method of immunomodulation of the invention may find particular utility in the transplant setting, i.e. the subject to be treated may be a transplant recipient.

As noted above, transplant patients typically require lifelong immunosuppression to prevent transplant rejection. Numerous immunosuppression drugs (sometimes termed “conditioning agents”) are used to induce donor specific tolerance and must be balanced with the need to minimize impairment of the host defences and avoid increasing the susceptibility to infection from all types of organisms.

There are two primary mechanisms for T cell immunosuppression: inhibition of the mTOR pathway and inhibition of the calcineurin/NFAT pathway. However, it has been found that Tregs are less susceptible to inhibition of the mTOR pathway than other T cells, such as cytotoxic T cells, i.e. Tregs are able to persist and function in the presence of mTOR inhibitors, such as everolimus.

The present inventors have determined that the use of low dose ATG as a conditioning agent may be particularly effective for subjects on calcineurin inhibitor (CNI) immunosuppression drug therapies, e.g. tacrolimus. In this respect, the profound and prolonged lymphodepletion caused by low dose ATG allows abrupt cessation of CNI administration shortly before administration of the medicament comprising Tregs, e.g. tacrolimus may be stopped 1 week before administration on the medicament. This ensures the administered Tregs are not suppressed and, advantageously, permits the administration of a single dose of Tregs, which reduces the burden on the patient and reduces the resources required for providing an effective immunomodulation treatment. Moreover, suppression of other immune cells, e.g. T cells, that might otherwise inhibit, antagonise or mask the therapeutic effect of the administered Tregs may be achieved with mTOR inhibitors, such as everolimus, without impacting significantly on the activity of the administered Tregs. As discussed further below, immunosuppression with an mTOR inhibitor (e.g. everolimus) may be continued after administration of the Tregs for sufficient time to allow the administered Tregs to have the therapeutic effect and/or to engraft.

Thus, in some embodiments, the subject to be treated is on an immunosuppressive drug therapy comprising a calcineurin inhibitor (CNI). CNIs include tacrolimus and cyclosporine. In some embodiments, the CNI is tacrolimus.

The immunosuppressive drug therapy may be a combination therapy, e.g. comprising or consisting of a CNI and an mTOR inhibitor. mTOR inhibitors include everolimus and sirolimus. In some embodiments the mTOR inhibitor is everolimus.

Thus, the immunosuppressive drug therapy may be a combination therapy comprising or consisting of tacrolimus and everolimus.

As it is preferred that the subject on an immunosuppressive drug therapy is on a combination therapy comprising or consisting of tacrolimus and everolimus, if the subject in need of treatment is on an immunosuppressive therapy other than a combination therapy consisting of tacrolimus and everolimus, it may be necessary to switch the immunosuppressive therapy of the subject. It will be appreciated that in order to ensure the subject is stable on the new immunosuppressive therapy, it may be advantageous to ensure that the step of switching is completed prior to the step of administering the low dose of ATG and/or prior to the step of obtaining Tregs from the subject, e.g. before the step of performing leukapheresis on the subject.

Accordingly, when the subject is on an immunosuppressive drug therapy other than a combination therapy consisting of tacrolimus and everolimus, the method may comprise a step of switching the immunosuppressive drug therapy to a combination therapy consisting of tacrolimus and everolimus.

The step of switching the immunosuppressive drug therapy to a combination therapy consisting of tacrolimus and everolimus may be completed before the step of administering the low dose of ATG, e.g. at least about 6 weeks before the step of administering the low dose of ATG, such as about 6-15 or 6-10 weeks before the step of administering the low dose of ATG.

Furthermore, the step of switching the immunosuppressive drug therapy to a combination therapy consisting of tacrolimus and everolimus may be completed before leukapheresis is performed on the subject to isolate T cells for use in the preparation of the medicament, e.g. at least about 2 weeks before leukapheresis is performed on the subject, such as about 2-10, 2-8 or 2-6 weeks before leukapheresis is performed on the subject.

As noted above, the use of low dose ATG advantageously facilitates the abrupt cessation of the administration of a CNI (e.g. tacrolimus) shortly after administration of the low dose of ATG and before administration of the Tregs. Accordingly, the method may comprise stopping (ceasing, cessation of, terminating) administration of a CNI (e.g. tacrolimus) after the step of administering the low dose of ATG and before administration of the medicament comprising Tregs. The step of stopping administration of a CNI (e.g. tacrolimus) may occur at any stage after the step of administering the low dose of ATG and before administration of the medicament comprising Tregs, but may be up to about 8 weeks, such as about 1-7 weeks, 2-6 weeks or 2-4 weeks, e.g. about 3 weeks, after the step of administering the low dose of ATG. Additionally or alternatively, the step of stopping administration of a CNI (e.g. tacrolimus) may occur up to about 14 days before administration of the medicament comprising Tregs, such as about 4-12 or 5-10 days before administration of the medicament comprising Tregs.

An objective of the immunomodulation therapy of the present invention in the context of transplant recipients is to reduce or eliminate the need for immunosuppressive drug therapies, such as CNIs and mTOR inhibitors, e.g. to achieve functional immune tolerance. In this respect, immunosuppressive drugs are associated with numerous negative side effects including nephrotoxicity, cancer, diabetes and cardiovascular disease, such as hypertension. Moreover, immunosuppressive drugs are also known to interact with many other drugs, which may impact on the treatment of other diseases or conditions.

Accordingly, the method may further comprise a step of weaning the subject off any immunosuppressive drugs that are still being administered after administration of the medicament comprising Tregs. As noted above, the subject may be receiving an mTOR inhibitor (e.g. everolimus) following the administration of the medicament comprising Tregs. Thus, the method may further comprise a step of weaning the subject off an mTOR inhibitor (e.g. everolimus) after administration of the medicament comprising Tregs.

In the context of the present invention the term “weaning” may be used interchangeably with the term “drug tapering”, which refers to the gradual discontinuation or reduction of a therapeutic dose of a particular drug (e.g. mTOR inhibitor, such as everolimus) over a period of time.

It will be appreciated that any suitable drug tapering protocol may be used to wean the subject off any immunosuppressive drugs that are still being administered after administration of the medicament comprising Tregs (e.g. mTOR inhibitor, such as everolimus) and is within the purview of the skilled person. For example, weaning may be started at least about 4 weeks after administration of the medicament comprising Tregs, such as about 4-12 weeks or 5-10 weeks after administration of the medicament comprising Tregs. The drug tapering may occur over any suitable time period, such as about 15-60 weeks or about 20-40 weeks, and may involve any suitable number of stepwise reductions in dose, e.g. 2-10 or 4-8 reductions. Moreover, each reduction in dose may be equal or the reduction in dose may be gradually increased or decreased over the drug tapering period.

In a representative example, the subject may be weaned off everolimus about 6 weeks after the step of administering the medicament comprising Tregs, wherein the dose is reduced by about 25% every 8 weeks until the weaning process is complete, i.e. everolimus administration ceases about 30 weeks after the step of administering the medicament comprising Tregs.

It will be understood that the drug tapering process may not result in complete cessation of the drug, i.e. tapering may stop before the dose of the immunosuppressive drug reaches 0. In other words, the subject may continue to be administered a reduced amount of the immunosuppressive drug (i.e. reduced compared to the dose prior to administration of the medicament comprising Tregs, i.e. prior to the immunomodulation therapy).

Alternatively, in some embodiments, the method may involve a step of stopping (i.e. abruptly and immediately ceasing or discontinuing) administration of any immunosuppressive drugs that are still being administered after administration of the medicament comprising Tregs. In other words, the method may not involve a drug tapering process for any immunosuppressive drugs (e.g. everolimus) that are still being administered after administration of the medicament comprising Tregs. Any suitable timing of cessation of the administration of everolimus may be used in the method of the invention, e.g. at least about 4 weeks after administration of the medicament comprising Tregs, such as about 4-12 weeks or 5-10 weeks after administration of the medicament comprising Tregs.

As noted above, in some embodiments the subject is a transplant recipient. Thus, the method of immunomodulation may be for treating and/or preventing rejection of a transplant, e.g. treating and/or preventing cellular and/or humoral transplant rejection. In further embodiments, the method of immunomodulation is for inducing functional immune tolerance in a transplant recipient. In still further embodiments, the method of immunomodulation is for treating and/or preventing graft-versus-host disease (GvHD).

The transplant may be from a living or deceased donor. Typically, the transplant is an allograft or allotransplantation, i.e. a transplant of an organ or tissue between two genetically non-identical members of the same species. However, the transplant may be a xenograft or xenotransplantation, i.e. a transplant of an organ or tissue from one species to another, e.g. a donor porcine heart valve transplanted into a human recipient.

The transplant may be a solid organ (i.e. a whole solid organ or a part thereof) or a graft. In particular, the solid organ may be a liver, kidney, heart, lung, pancreas, intestine or stomach. The intestine typically is small intestine or a part thereof. Thus, the transplant may be a part of a solid organ, such as a portion of a liver (e.g. a portion of the right lobe, such as about 50-70% of the liver of a living donor), a heart valve or a lung lobe. In some embodiments, the transplant is a liver transplant, e.g. a whole liver or a portion of a liver.

A graft typically refers to tissues or cells, i.e. a tissue or cell transplant. In particular, the tissue may be a vascularized composite tissue, skin, a cornea, a blood vessel, a muscle, a heart valve or a bone (e.g. an arm or leg bone). The cells may be islet of Langerhans cells (pancreas islet cells), bone marrow or adult stem cells.

A vascularized composite tissue graft refers to a graft that is composed of multiple different tissues that are transplanted together as a single unit. A typical example is a hand graft, which consists of muscles, skin, bone, vessels, and nerves. Thus, in some embodiments the transplant is a limb, such as a hand or foot.

Rejection of a transplant or transplant rejection refers to immune-mediated rejection of the transplant (i.e. allograft). Rejection may be hyperacute rejection, acute rejection or chronic rejection. Rejection may result in numerous symptoms including abnormal organ function, malaise, anorexia, muscle ache, low fever, increase in white blood count, and graft-site tenderness. In a representative embodiment, liver rejection may present as elevated levels of markers, such as AST, ALT, GGT; abnormal liver function values such as prothrombin time, ammonia level, bilirubin level, albumin concentration; and abnormal blood glucose. Physical symptoms associated with liver transplant rejection may include encephalopathy, jaundice, bruising and bleeding tendency.

Hyperacute rejection is caused by preformed anti-donor antibodies. It is characterized by the binding of these antibodies to antigens on donor tissue, e.g. vascular endothelial cells. Complement activation is involved and the effect is usually rapid and profound, with rejection occurring within minutes to hours after the transplant procedure.

Acute rejection is mediated by T cells and involves direct cytotoxicity and cytokine mediated pathways. Acute rejection is the most common form of rejection and the primary target of immunosuppressive agents. Acute rejection is usually seen within days or weeks of the transplant.

Chronic rejection is the presence of any sign and symptom of rejection after one year. The cause of chronic rejection is still unknown, but an acute rejection is a strong predictor of chronic rejections.

Thus, the method of treating and/or preventing rejection of a transplant may be for treating and/or preventing acute or chronic rejection of a transplant, particularly an acute rejection of a transplant (e.g. liver transplant). Alternatively viewed, the method of treating and/or preventing rejection of a transplant may be for treating and/or preventing cellular transplant rejection, i.e. transplant rejection mediated by T cells.

The term “functional immune tolerance” refers to the capacity to remove (eliminate, cease or discontinue) or reduce immunosuppressive drug therapies without rejection of the transplant for a prolonged period of time (e.g. at least about 6 months, such as at least about 9 months or 12 months). Thus, inducing functional immune tolerance refers to a treatment that may allow full removal of one or more (e.g. all) immunosuppressants (e.g. full tolerance, also known as operational immune tolerance, particularly in the context of liver transplants) or a reduction in the dose and/or number of immunosuppressants being administered to the subject. Removal (cessation, discontinuation or elimination) of an immunosuppressant may be performed using any suitable protocol. For instance, a drug tapering protocol may be used to reduce the dose of one or more immunosuppressive drugs, e.g. everolimus, as described above. If the subject is receiving more than one immunosuppressive drug, removal (e.g. tapering) may be performed separately for each drug or removal (e.g. tapering) may be performed for multiple (e.g. all) drugs concurrently. Moreover, one or more immunosuppressive drugs may be reinstated (following complete cessation) or the dose increased (following tapering to a reduced dose) upon signs of transplant rejection.

Thus, “without rejection of the transplant” means that the transplant (e.g. solid organ, such as a liver) shows stable function and/or no histopathologic evidence of rejection (e.g. cellular transplant rejection) for a prolonged period of time (e.g. at least about 6 months, such as at least about 9 months or 12 months). Alternatively viewed, the subject (i.e. transplant recipient) shows no signs associated with transplant rejection, e.g. chronic or acute rejection. The skilled person readily could determine whether a transplant shows stable function and/or no histopathologic evidence of rejection using routine methods known in the art.

As noted above, immunosuppressive drugs are associated with several negative side effects including nephrotoxicity, cancer, diabetes and cardiovascular disease, such as hypertension. Thus, even a small reduction in the dose of immunosuppressant drugs required by transplant recipients may be beneficial, insofar as it may reduce the risk of a subject developing these disorders or conditions or even prevent the subject from developing these disorders or conditions.

Thus, the method of inducing functional immune tolerance (e.g. operational immune tolerance) may be viewed as:
(a) reducing the risk of a subject developing; or
(b) preventing a subject from developing,
nephrotoxicity, cancer, diabetes and/or cardiovascular disease,
wherein the subject is on an immunosuppressive drug therapy (e.g. a CNI and/or mTOR inhibitor).

Alternatively viewed, the invention may be seen as providing a method of:
(a) reducing the risk of a subject developing; or
(b) preventing a subject from developing,
nephrotoxicity, cancer, diabetes and/or cardiovascular disease,
wherein the subject (e.g. a transplant recipient) is on an immunosuppressive drug therapy (e.g. a CNI and/or mTOR inhibitor), the method comprising:
(a) administering a low dose of anti-thymocyte globulin (ATG) to the subject; and
(b) administering a medicament comprising regulatory T cells (Tregs) to the subject,
wherein step (a) occurs about 8 weeks or less before step (b).

In a further embodiment, the invention may provide a low dose of anti-thymocyte globulin (ATG) for use in:
(a) reducing the risk of a subject developing; or
(b) preventing a subject from developing,
nephrotoxicity, cancer, diabetes and/or cardiovascular disease,
wherein the subject (e.g. a transplant recipient) is on an immunosuppressive drug therapy (e.g. a CNI and/or mTOR inhibitor) and is administered a medicament comprising regulatory T cells (Tregs), wherein said low dose of ATG is for administration about 8 weeks or less before administration of said medicament.

It will be appreciated that the low dose of ATG and medicament comprising Tregs may be provided as a combined therapy or product or a kit for separate and sequential use as described above.

In view of the potential utility of Tregs to suppress inflammation and autoimmune responses, it will be appreciated that the invention extends to the treatment of subjects with inflammatory or autoimmune diseases of conditions. While this includes the treatment of transplant recipients (e.g. liver transplant recipients), who may suffer from undesired inflammatory responses (e.g. cell mediated transplant rejection), it is not limited to this aspect.

Thus, in some embodiments, the subject to be treated has an inflammatory or autoimmune disease or condition. Alternatively viewed, the invention provides a method of treating an inflammatory or autoimmune disease or condition in a subject in need thereof, i.e. the method of immunomodulation is for treating an inflammatory or autoimmune disease or condition. Thus, in some embodiments, the subject is not a transplant recipient and/or is not on an immunosuppressive therapy.

Thus, the disease, condition or disorder to be treated may be an inflammatory or autoimmune disease, condition or disorder. Thus, the invention may find utility in treating and/or preventing (e.g. reducing the risk of) an inflammation or an autoimmune disease or disorder. The disease may be chronic or acute, preferably chronic. The inflammatory or autoimmune disease may be associated with the gastrointestinal tract (e.g. large and/or small intestine), skin, lung or liver. In one aspect, the inflammatory disease is inflammatory bowel disease. In another aspect, the inflammatory disease is transplant rejection (e.g. solid organ transplant rejection, such as liver transplant rejection).

Thus, the inflammatory or autoimmune disease or disorder may be selected from inflammatory bowel disease (including inflammation of the gastrointestinal tract), diabetes (e.g. type I diabetes), transplant rejection (e.g. organ or graft rejection), rheumatoid arthritis, multiple sclerosis, atherosclerosis, asthma (e.g. allergic asthma), rhinitis (e.g. allergic rhinitis), graft versus host disease, inflammatory lung disease (e.g. COPD), inflammatory skin disease and inflammatory liver disease.

Inflammatory Bowel Disease (IBD) consists of two major types, namely Crohn's Disease (CD) and Ulcerative Colitis (UC). CD affects any part of the gastrointestinal tract, from mouth to anus, although in the majority of the cases the disease starts in the distal small bowel. CD involves the whole bowel wall (transmural inflammation). UC is restricted to inflammation in the colon and involves only the mucosa.

Common symptoms associated with IBD are abdominal pain, vomiting, diarrhoea, rectal bleeding, weight loss and cramps or spams in the lower abdomen. In severe cases the tendency to develop intra-abdominal fistulas gives rise to deep infections.

Thus, IBD to be treated or prevented according to the present invention may be selected from Crohn's Disease and Ulcerative Colitis.

As used herein, the term “preventing” includes providing prophylaxis with respect to occurrence or recurrence of a particular disease, disorder, or condition in an individual. An individual may be predisposed to, susceptible to a particular disease, disorder or condition, or at risk of developing such a disease, disorder, or condition, but has not yet been diagnosed with the disease, disorder, or condition.

As used herein, an individual “at risk” of developing a particular disease, disorder or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder or condition, as known in the art. An individual having one or more of these risk factors has a higher probability of developing a particular disease, disorder or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum amount required to affect a measurable improvement of a particular disease, disorder or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the medicament and/or pharmaceutical compositions are outweighed by the therapeutically beneficial effects.

The terms “subject”, “patient” and “individual” are used interchangeably herein and refer to a mammal, preferably a human. In particular, the terms subject, patient and individual refer to a human having a disease or disorder as defined herein in need of treatment. In particular embodiments, the subject is a transplant recipient (e.g. liver transplant recipient) and/or is on an immunosuppressive drug therapy, particularly comprising a CNI.

The patient may be subjected to other treatments prior to, contemporaneously with, or after the treatments of the present invention. For instance, the patient may be treated with other procedures for the treatment of symptoms associated with the disease or disorder.

The medicament and/or pharmaceutical composition described above may be administered in combination with other therapeutic agents for the treatment of symptoms associated with the disease or disorder or other underlying condition.

Thus, the medicament and/or pharmaceutical composition described above may contain one or more additional therapeutic agents or may be for administration with one or more additional therapeutic agents.

The medicament and/or pharmaceutical composition may contain or be administered with a further therapeutic agent useful in treating an inflammatory or autoimmune disease as defined herein, e.g. inflammatory bowel disease, transplant rejection.

The other therapeutic agents may be part of the same medicament comprising the Treg cell or cell population, in the form of a mixture, wherein the cell or cell population and the other therapeutic agent are intermixed in or with the same pharmaceutically acceptable solvent and/or carrier or may be provided separately as part of a separate composition, which may be offered separately or together in form of a kit of parts.

Thus, the medicament and/or pharmaceutical composition of the invention may be administered concomitantly with the other therapeutic agent separately, simultaneously or sequentially. For example, the medicament or pharmaceutical composition of the invention may be administered simultaneously with a first additional therapeutic agent or sequentially after or before administration of said first additional therapeutic agent. If the treatment regimen or schedule utilizes more than one additional therapeutic agent, the various agents may be partially administered simultaneously, partially sequentially in various combinations. For instance, where the subject is on an immunosuppressive drug therapy, the other or additional therapeutic agent is one or more immunosuppressive drugs, e.g. a CNI and/or mTOR inhibitor, such as tacrolimus and/or everolimus. Typically, these drugs are administered separately to the medicament and pharmaceutical composition described herein.

The therapeutic agents for use in combination with the medicament and/or pharmaceutical composition of the invention may be provided in pharmaceutical compositions as defined above and may be administered as defined above. Thus, the compositions comprising additional therapeutic agents may comprise pharmaceutically acceptable excipients, solvents and diluents suitable for such formulations.

The skilled person will be aware of suitable dosage ranges for any given additional therapeutic agent. Suitably, the additional therapeutic agent is present in the medicament and/or pharmaceutical composition, or administered to the subject, in its typical dose range.

While the skilled person readily could determine which embodiments described above may be combined, some representative embodiments of the invention are presented below.

In one aspect, the invention provides a method for conditioning a transplant recipient (e.g. a liver transplant recipient) on an immunosuppressive drug therapy comprising a CNI (e.g. tacrolimus) for treatment with a medicament comprising Tregs (e.g. engineered Tregs, particularly CAR-Tregs), the method comprising administering a low dose of ATG (e.g. about 3 mg/kg or less, optionally in a single dose) to the subject about 8 weeks or less (e.g. about 6 weeks) before administration of the medicament.

In another aspect, the invention provides a method of: (a) treating and/or preventing rejection (e.g. acute rejection) of a transplant (e.g. a liver transplant) in a subject on an immunosuppressive drug therapy comprising a CNI (e.g. tacrolimus); or (b) inducing functional immune tolerance in a transplant recipient (e.g. a liver transplant recipient) on an immunosuppressive drug therapy comprising a CNI (e.g. tacrolimus),
the method comprising:
(a) administering a low dose of ATG to the subject/transplant recipient (e.g. about 3 mg/kg or less, optionally in a single dose); and
(b) administering a medicament comprising Tregs to the subject (e.g. engineered Tregs, particularly CAR-Tregs, optionally in a single dose),
wherein step (a) occurs about 8 weeks or less (e.g. about 6 weeks) before step (b).

In some embodiments, the immunosuppressive drug therapy is a combination therapy consisting of a CNI and an mTOR inhibitor. In a particular aspect, the immunosuppressive drug therapy is a combination therapy consisting of tacrolimus and everolimus.

Moreover, the method may comprise stopping administration of tacrolimus about 2-4 weeks after the step of administering the low dose of ATG and/or about 5-10 days before administration of the medicament. Furthermore, the method may comprise a step of weaning the subject off everolimus. In a particular aspect, weaning is started at least about 4 weeks after administration of the medicament.

In the methods described above, the engineered Treg may comprise a chimeric receptor (e.g. a CAR) that selectively binds to a HLA antigen present in the transplanted organ (e.g. liver) but not in the patient, an organ-specific, tissue-specific or cell-specific antigen (e.g. a liver-specific antigen such as NTCP), or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

It will be appreciated that the low dose of ATG and medicament comprising Tregs may be provided as a combined therapy or product or a kit for separate and sequential use as described in the exemplary methods above.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### EXAMPLES

### Example 1

### Clinical Trial Protocol

This study evaluates the safety and preliminary clinical activity of an autologous CAR-T regulatory (CAR-Treg) cell treatment in subjects that have received a liver transplant. The CAR is specific for HLA-A2, which localizes the activity of the CAR-Tregs to the site of the transplanted organ in HLA-A2 mismatch liver transplant patients (i.e. HLA-A2 negative recipients who received an HLA-A2 positive donor liver).

Eligible subjects will be enrolled into the study and switched onto a standardised immunosuppression regimen of Tacrolimus (TAC) and Everolimus (EVR), if not already on this regimen. Two weeks after conversion to the standardised immunosuppression regimen, subjects will undergo leukapheresis to obtain Treg cells. These Treg cells will be transduced with the HLA-A2 CAR to manufacture the autologous CAR-Treg cell treatment.

Four weeks prior to receiving the CAR-Treg cell treatment, each subject will be administered a lymphodepleting regimen of rATG.

Tacrolimus will be stopped for all subjects within 1 week prior to the infusion to avoid negative impact on Treg cells. A target dose of the CAR-Treg cell treatment (i.e., of transduced Tregs) will be administered by intravenous infusion at a specified flow rate.

Six weeks post infusion, subjects will be weaned off Everolimus by 25% every 8 weeks until the weaning process is complete, i.e. Everolimus administration ceases about 30 weeks after the step of administering the CAR-Treg cell treatment.

Subjects will be monitored to determine if they can successfully achieve operational tolerance (defined in this case as 1 year free from immunosuppressants with stable liver tests and liver histology and in absence of graft loss or patient death).

1. A method for conditioning a subject for treatment with a medicament comprising regulatory T cells (Tregs), the method comprising administering a low dose of anti-thymocyte globulin (ATG) to the subject about 8 weeks or less before administration of the medicament.

2. A method of immunomodulation of a subject in need thereof comprising the steps of:
(a) administering a low dose of anti-thymocyte globulin (ATG) to the subject; and
(b) administering a medicament comprising regulatory T cells (Tregs) to the subject,
wherein step (a) occurs about 8 weeks or less before step (b).

3. The method of claim 1 or 2, wherein the Tregs are chimeric antigen receptor (CAR)-Tregs.

4. The method of any one of claims 1 to 3, wherein the low dose of ATG is administered in a single dose.

5. The method of any one of claims 1 to 4, wherein the subject does not receive any further doses of ATG after administration of the medicament.

6. The method of any one of claims 1 to 5, wherein the subject is administered a single dose of the medicament.

7. The method of any one of claims 1 to 6, further comprising a step of performing leukapheresis on the subject to isolate T cells for use in the preparation of the medicament before the step of administering the low dose of ATG.

8. The method of any one of claims 1 to 7, wherein the ATG is rabbit ATG.

9. The method of any one of claims 1 to 8, wherein the low dose of ATG is about 3 mg/kg or less.

10. The method of any one of claims 1 to 9, wherein the subject is on an immunosuppressive drug therapy comprising a calcineurin inhibitor (CNI), optionally wherein the CNI is tacrolimus.

11. The method of claim 10, wherein the immunosuppressive drug therapy is a combination therapy consisting of a CNI and an mTOR inhibitor, optionally wherein the immunosuppressive drug therapy is a combination therapy consisting of tacrolimus and everolimus.

12. The method of claim 10 or 11, wherein when the subject is on an immunosuppressive drug therapy other than a combination therapy consisting of tacrolimus and everolimus, the method comprises a step of switching the immunosuppressive drug therapy to a combination therapy consisting of tacrolimus and everolimus.

13. The method of claim 12, wherein the step of switching the immunosuppressive drug therapy to a combination therapy consisting of tacrolimus and everolimus is complete before the step of administering the low dose of ATG, preferably at least about 6 weeks before the step of administering the low dose of ATG.

14. The method of claim 12 or 13, wherein the step of switching the immunosuppressive drug therapy to a combination therapy consisting of tacrolimus and everolimus is complete before leukapheresis is performed on the subject to isolate T cells for use in the preparation of the medicament, preferably at least about 2 weeks before leukapheresis is performed on the subject.

15. The method of any one of claims 10 to 14, wherein the method comprises stopping administration of tacrolimus about 2-4 weeks after the step of administering the low dose of ATG and/or about 5-10 days before administration of the medicament.

16. The method of any one of claims 12 to 15 further comprising a step of weaning the subject off everolimus, optionally wherein weaning is started at least about 4 weeks after administration of the medicament.

17. The method of any one of claims 1 to 16, wherein the subject has an inflammatory or autoimmune disease or condition.

18. The method of any one of claims 2 to 17, wherein the method of immunomodulation is for treating an inflammatory or autoimmune disease or condition.

19. The method of claim 17 or 18, wherein the inflammatory or autoimmune disease or condition is type I diabetes or inflammatory bowel disease.

20. The method of any one of claims 1 to 19, wherein the subject is a transplant recipient.

21. The method of any one of claims 2 to 20, wherein the method of immunomodulation is for treating and/or preventing rejection of a transplant.

22. The method of any one of claims 2 to 20, wherein the method of immunomodulation is for inducing functional immune tolerance in a transplant recipient.

23. The method of any one of claims 20 to 22, wherein the transplant is a solid organ or part thereof or a graft.

24. The method of claim 23, wherein the solid organ is a liver, kidney, heart, lung, pancreas, intestine or stomach or a part thereof, optionally a portion of a liver, a heart valve or a lung lobe.

25. The method of claim 23, wherein the graft is a cell or tissue transplant, optionally wherein the tissue a vascularized composite tissue, skin, a cornea, a blood vessel, a muscle, a heart valve or a bone and/or the cells are islet of Langerhans cells, bone marrow or adult stem cells.

26. A low dose of anti-thymocyte globulin (ATG) for use in conditioning a subject for treatment with a medicament comprising regulatory T cells (Tregs), wherein said ATG is for administration about 8 weeks or less before administration of the medicament.

27. A low dose of anti-thymocyte globulin (ATG) for use in immunomodulation of a subject in need thereof, wherein said immunomodulation comprises administration of a medicament comprising regulatory T cells (Tregs) to the subject and wherein said low dose of ATG is for administration about 8 weeks or less before administration of said medicament.

28. A combination therapy or product comprising a low dose of anti-thymocyte globulin (ATG) and a medicament comprising regulatory T cells (Tregs), wherein the components of the combination therapy or product are for separate and sequential use in immunomodulation of a subject, wherein said ATG is for administration about 8 weeks or less before administration of the medicament.

29. A kit comprising:
(i) anti-thymocyte globulin (ATG); and
(ii) a medicament comprising regulatory T cells (Tregs),
for separate and sequential use in the immunomodulation of a subject, wherein said ATG is for administration at a low dose about 8 weeks or less before administration of the medicament.

30. A low dose of anti-thymocyte globulin (ATG) for use of claim 26 or 27, a combined therapy or product for use of claim 28 or a kit for use of claim 29, wherein the ATG, medicament, subject or immunomodulation is as defined in any one of claims 3 to 25.
